(19) United States
(12) Patent Application Publication
(10) Pub. No.: US 2004/0148193 A1
(43) Pub. Date: Jul. 29, 2004

(54) Method, system, and program for managing patient biometric data from patients in a health care environment

(75) Inventor: Justin C. Blackburn, Surprise, AZ (US)

Correspondence Address: KONRAD RAYNES VICTOR & MANN LLP, Suite 210, 315 S. Beverly Drive, Beverly Hills, CA, 90212, US

(73) Assignee: International Business Machines Corporation

(21) Appl. No.: 10/349,988

(22) Filed: Jan. 23, 2003

(51) Int. Cl. G06F 17/60; A61B 5/00

(52) U.S. Cl. 705/2; 600/300

(57) ABSTRACT

Provided are a method, system, and program for managing patient biometric data from a plurality of patients in a health care environment. Different types of biometric data are received from a plurality of different types of medical monitoring devices. A determination is made from display settings one of a plurality of display monitors on which to display received biometric data from one of the plurality of medical monitoring devices. The received biometric data is displayed on the determined monitor, wherein each monitor is capable of displaying biometric data from at least one patient and from at least one monitoring device.

### BACKGROUND OF THE INVENTION

1. Field of the Invention

The present invention relates to a method, system, and program for managing patient biometric data from patients in a health care environment.

2. Description of the Related Art

In a hospital environment, such as an Intensive Care Unit (ICU), a nurse or other medical practitioner may monitor biometric data from different patients at a central station. Typically, different types of monitoring equipment are used to measure different biometric data, such blood-sugar levels, blood oxygenation, blood pressure, pulse, electrocardiogram (EKG), etc. Each specific piece of monitoring equipment in each patient room may be hard wired to a monitor at the nurse station such that one monitor will display the particular type of bio-feedback for multiple patients from the same vendor machines in the patients' rooms. Often, there will be multiple monitors, one for each type of monitor device, to provide a particular type of bio-feedback for all patients hooked-up to the particular device. Further, in many nurse stations, displays are grouped by type, such as all the EKG displays are in one area and all the respirator screens in another. The health care personnel will thus have to scan different screens to observe different biometric data for the same patient.

Further complicating patient monitoring is that different monitoring devices often use proprietary interfaces that provide a direct link from the monitoring device in the patient room to the display monitor at the nurse station. This means that the hospital must use the specific display monitor provided by the monitoring equipment vendor even if a monitor for another type of monitoring equipment is available for use. The proprietary nature of medical monitoring equipment complicates the ability to integrate and manage the machines together.

For these reasons, there is a need in the art to provide improved techniques for managing biometric data from multiple patients using different types of medical monitoring devices.

### SUMMARY OF THE DESCRIBED IMPLEMENTATIONS

Provided are a method, system, and program for managing patient biometric data from a plurality of patients in a health care environment. Different types of biometric data are received from a plurality of different types of medical monitoring devices. A determination is made from display settings one of a plurality of display monitors on which to display received biometric data from one of the plurality of medical monitoring devices. The received biometric data is displayed on the determined monitor, wherein each monitor is capable of displaying biometric data from at least one patient and from at least one monitoring device.

In further implementations, the different types of medical monitoring devices may comprise medical devices that monitor different types of biometric data. Still further, the different types of medical monitoring devices may comprise medical devices from different medical device vendors.

In still further implementations, information for the display monitors is provided indicating at least one patient and at least one monitoring device for one display monitor, wherein displaying the received biometric data comprises displaying on the display monitor the biometric data from the at least one monitoring device and for the at least one patient indicated for the display monitor.

In yet further, a determination is made as to whether the received biometric data satisfies a threshold related to an alert and an alert action related to the threshold is performed if the received biometric data satisfies the threshold.

The described implementations provide improved techniques for integrating biometric data received from different devices gathering biometric data from one or more patients. Described implementations further provide techniques for integrating the management of multiple bio-feedback monitoring devices that may be monitoring patients in a health care facility.

### BRIEF DESCRIPTION OF THE DRAWINGS

Referring now to the drawings in which like reference numbers represent corresponding parts throughout:

FIG. 1 illustrates a health care computing environment in which aspects of the invention are implemented;

FIGS. 2, 3, and 4 illustrate data structures used to maintain biometric data and information to manage biometric data in accordance with implementations of the invention;

FIG. 5 illustrates operations performed to generate alerts for patient biometric data in accordance with implementations of the invention;

FIG. 6 illustrates operations performed to enable a user to configure display settings for received biometric data in accordance with implementations of the invention;

FIG. 7 illustrates operations performed to display received biometric data in accordance with implementations of the invention; and

FIG. 8 illustrates an architecture of computing components in the health care environment of FIG. 1.

### DETAILED DESCRIPTION OF THE PREFERRED EMBODIMENTS

In the following description, reference is made to the accompanying drawings which form a part hereof and which illustrate several embodiments of the present invention. It is understood that other embodiments may be utilized and structural and operational changes may be made without departing from the scope of the present invention.

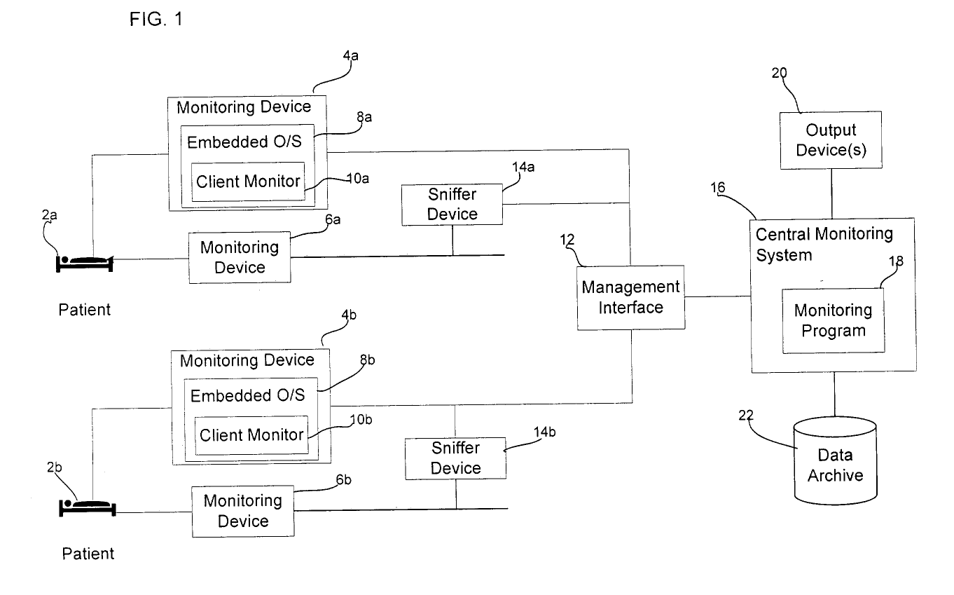

FIG. 1 illustrates a health care computing environment in which aspects of the invention may be implemented. Biometric data from one or more hospital patients 2a, 2b is monitored by one or more monitoring devices 4a, 4b and 6a, 6b. The monitoring devices may comprise any type of medical device known in the art that is used to measure patient bodily operations and levels, such as an EKG machine, blood pressure, blood oxygen level, blood sugar level, etc. Certain of the monitoring devices, such as devices 2a, 2b include an embedded operating system 8a, 8b, such as Windows CE, Linux, etc. Those devices that include an embedded operating system 8a, 8b further include a client monitor program 10a, 10b to communicate biometric data and events to the management interface 12 system. In this way, the management interface system 12 consolidates data from one or more client monitor 10a, 10b programs. The client monitor 10a, 10b may communicate with the management interface 12 via the network interface included in the monitoring device 4a, 4b, such as via a TCP/IP connection, Bluetooth connection, etc.

The hospital network environment may further include monitoring devices 6a, 6b that do not include an embedded operating system. Such monitoring devices 6a, 6b may instead include special purpose or proprietary software and/or hardware to communicate measured biometric data to an external device. In the described implementations, a sniffer device 14a, 14b intercepts data transmitted from the monitoring device 6a, 6b, and then transfers such transmitted biometric data to the management interface 12. The sniffer device 14a, 14b may comprise a separate hardware component designed to read and process data transmitted from the monitoring device 6a, 6b. For instance, the monitoring devices 6a, 6b may be linked via a cable or other connection line, including wireless connections, to the sniffer device 14a, 14b. The sniffer device 14a, 14b thus intercepts data the monitoring device 6a, 6b is transmitting to a display monitor, which may involve a proprietary transmission format. In the health care environment of FIG. 1, the display monitor provided by the vendor of the monitoring device 6a, 6b may not be used because the. data is displayed according to the described implementations. The sniffer device 14a, 14b may be programmed to process the data transmitted from the monitoring device 6a, 6b and transform such data to a format compatible with the patient data fonnats used by other downstream components, such as the management interface 12 and monitoring program 18, discussed below. The sniffer devices 14a, 14b may comprise a special-purpose hardware component or include an embedded operating system and a client monitor program to communicate data to the management interface 12.

In certain implementations, the management interface system 12 transmits data from the different monitoring devices 4a, 4b, 6a, 6b to a central monitoring system 16. The central monitoring system 16 runs a monitoring program 18 that processes the biometric data from the different monitoring devices to display on one or more output devices 20, such as display monitors. In this way data from different monitoring devices, which may include devices with proprietary hardware and/or software, are gathered and transmitted to a central monitoring system 16, which can be configured to further process and output the biometric data from the different devices according to user specified commands.

The client monitor programs 10a, 10b, management interface system 12 and central monitoring system 16 may be part of an integrated product that allows for the monitoring of data and events at distributed devices, such as the International Business Machines (IBM®) Tivoli® Monitoring program, described in the IBM publication “IBM Tivoli Monitoring User's Guide, Version. 5.1.1”, IBM document no. SH19-4569-01 (IBM Copyright 1999, 2002), which publication is incorporated herein by reference in its entirety, or any other program known in the art capable of monitoring data and events at distributed devices.

The monitoring program 18 may provide a management interface accessible to users over a network, such as accessible to Web type interfaces, e.g., Hypertext Markup Language (HTML) browsers, to allow users to access the data gathered at the central monitoring system 16 and configure settings of the monitoring program 18. The monitoring program 18 may further archive data received from the different monitoring devices 4a, 4b, 6a, 6b in a data archive 22, which may comprise any non-volatile storage system known in the art. The monitoring program 18 may be configured to transmit paging alerts or electronic mail to a health care personnel in response to specific events being detected from one or more of the monitoring devices. Further, the arrangement and presentation of data on the one or more output devices 20 may be configured through the monitoring program 18. For instance, a user can configure to display all the biometric for one patient gathered from multiple monitoring devices to be displayed on a single display monitor and configure to display biometric data from multiple patients from one or more monitoring devices on one monitoring device.

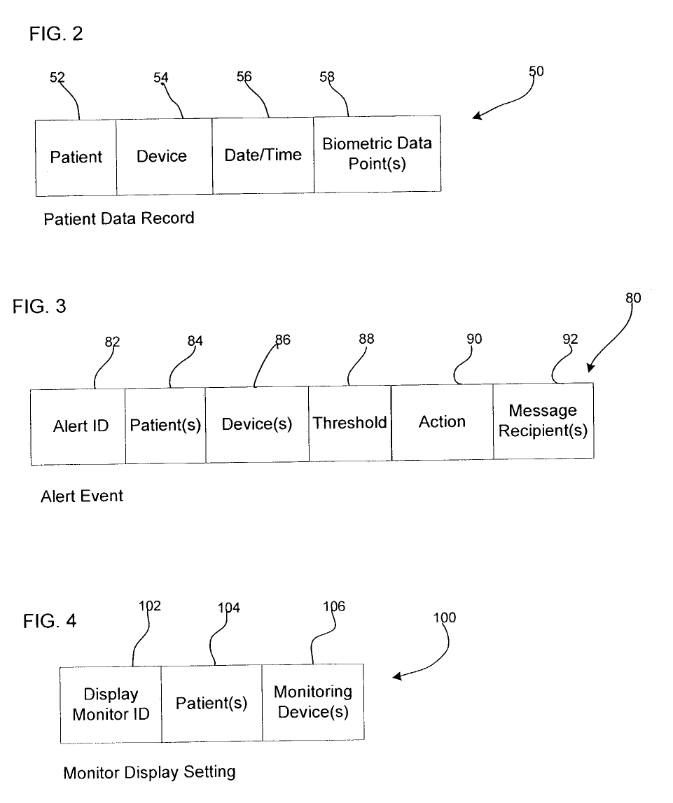

FIG. 2 illustrates a patient data record 50 that includes biometric data for a patient from a particular monitoring device 4a, 4b, 6a, 6b. Either the management interface 12, the client monitor 10a, 10b or the sniffer devices 14a, 14b may include data received for a patient from a particular monitoring device as a patient data record 50. Further, the patient data records may be archived in the data archive 22 and available for later review and analysis. Each patient data record 50 may include the following information:

patient 52: unique identifier of patient for which data is received. May also indicate all patients, or patients from a certain area of the health care facility, such as a wing of suites, floor of the building, a room, hall, etc.

device 54: identifies the monitoring device that gathered the patient biometric data. This identification may indicate a vendor and product type, or also include a unique product identifier, such as a serial number.

date/time 56: indicates a date and time when the data was gathered.

biometric data point(s) 58: the one or more biometric data points gathered at the specified data and time.

FIG. 3 illustrates an alert event 80 which includes configurable information a user may enter through the central monitoring system 16 to configure the monitoring program 18 to handle specific events. An alert event 80 may include the following information:

alert identifier (ID) 82: unique identifier of the alert.

patient(s) 84: the one or more patients to which the alert pertains. The patient may identify an individual or a room or bed or monitoring device number.

device(s) 86: the one or more monitoring devices to which the alert pertains.

threshold(s) 88: one or more thresholds for data received from the indicated monitoring device(s) that when satisfied trigger an alert. The threshold may occur if a specified threshold is exceeded or is not exceeded or met.

action(s) 90: the one or more actions that are performed if the alert is satisfied. For instance, if a patient's vital signs fall below a certain threshold, the action may comprise an emergency warning that is sounded to alert doctors and health personnel to immediately proceed to the patient to provide assistance. Alternatively, the action may comprise an alert or message displayed on the display monitor 20 on which the data is being displayed, or an e-mail or pager message to a person.

message recipient(s) 92: if the action indicates to send a message to notify certain health care providers of the threshold being satisfied, then the message recipients are those persons who are notified of the alert electronically, through paging, electronic mail, telephone call, etc.

FIG. 4 illustrates configurable monitor display settings 100 that a user may enter through a user interface of the monitoring program 18 to configure how gathered biometric data is displayed on a specified monitor. This configuration user interface may include drop down menus to enable selections of a display monitor and the patient(s) and monitoring device(s)s whose data will be displayed on the selected display. The monitor display settings 100 for a monitor may include:

monitor identifier 102: uniquely identifies one output device or display monitor in communication with the central monitoring system 16.

patient(s) 104: identifies one or more patients whose biometric data will be displayed at the identified monitor. The patient field may identify an individual or a grouping of one or more patients, e.g., patients on a floor, in a wing of a floor, building, room, bed, etc.

monitoring device(s) 106: identifies one or more devices whose data will be displayed at the identified monitor.

The patient data records 50, alert events 80, and monitor display settings 100 may be stored in a computer readable medium accessible to the monitoring program 18, or any other device in the health care environment that utilizes the information in data structures 50, 80, and 100.

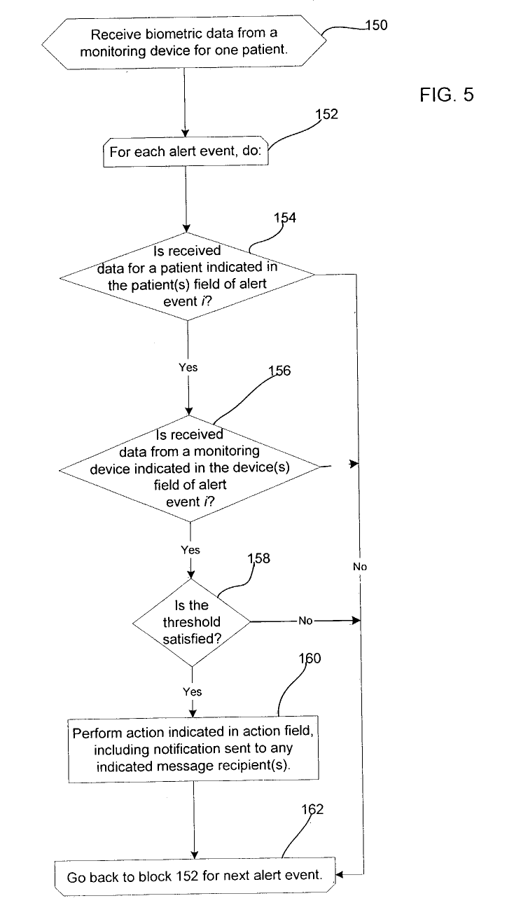

FIGS. 5, 6, and 7 illustrate operations performed by the monitoring program 18 to manage patient biometric data gathered by various monitoring devices in accordance with implementations of the invention. FIG. 5 illustrates operations performed by the monitoring program 18 determine whether one of the alert events is relevant to a received patient data record 50. Upon receiving (at block 150) biometric data from a monitoring device 4a, 4b, 6a, 6b in the form of a patient data record 50, the monitoring program 18 performs a loop at blocks 152 through 162 to check whether any of the alert events 80 are applicable to the received patient data record 50. If (at block 154) the received data is for a patient indicated in the patient(s) field 84 (where the patient may be specifically identified or a member of a group of patients indicated in the field 84) of alert event i and if (at block 156) the received data is from a monitoring device 4a, 4b, 6a, 6b indicated in the monitoring device field 86 of alert event i and if (at block 158) the received biometric data 58 satisfies the threshold 88, then the monitoring program 18 performs (at block 160) the one or more actions indicated in the action field 90, which may include notifying a person indicated in the message recipient field 92 of the alert. Alternatively, the alert action may cause some operation to be performed, such as automatically adjusting medication or assistance being provided to the patient, i.e., remotely increasing or decreasing the flow of medication. If the alert event i is not applicable to the received data, such as the case if “no” is returned at either of blocks 154, 156, and 158, or after performing the specified action at block 160, control proceeds (at block 162) back to block 152 to determine the applicability of any further alert events 80. One or more alert events may be applicable to a received biometric data.

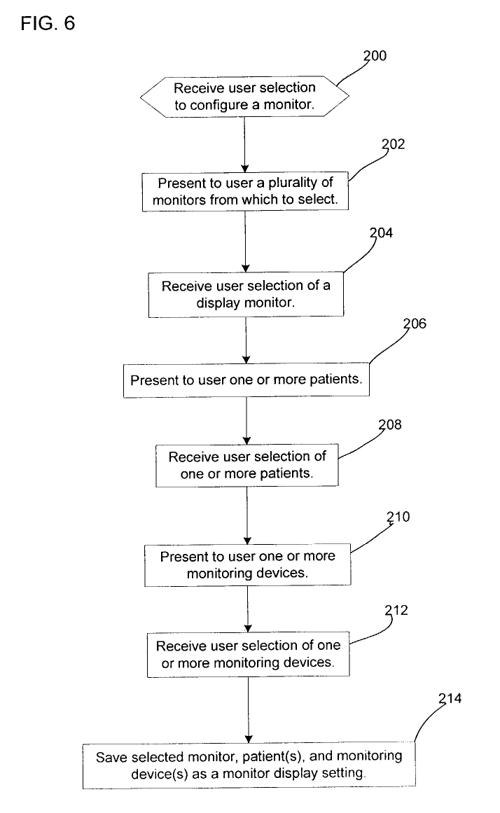

FIG. 6 illustrates operations performed by the monitor program 18 to allow a user to set a monitor display setting 100 to specify how data from one or more patients 2a, 2b and monitoring devices 4a, 4b, 6a, 6b is to be rendered on a specified display monitor. One or more monitor display settings 100 may be maintained for each output or display device 20 that may be controlled by the monitoring program 18. Upon receiving (at block 200) user selection to configure a display monitor (or other output device such as printer, remote display device, such as a computing device used by another health care worker, etc.), the monitor program 18 presents (at block 202) the user with a plurality of display monitors 20 to configure. Upon receiving (at block 204) user selection of a particular display monitor, the user is presented (at block 206) with one or more patients or grouping of patients (e.g., patients on a floor, wing of a floor, building, room, etc.) from which to select. Upon receiving (at block 208) user selection of one or more patient's (or group of patients) whose data will be displayed on the monitor selected at block 204, the monitoring program 18 presents (at block 210) the user with one or more monitoring device types (or specific devices) whose data will be displayed in the selected display monitor 20. Upon receiving (at block 212) user selection of one or more monitoring devices, the selected monitor 20, patient(s) 2a, 2b, and monitoring device(s) are stored as monitor display setting 100 information. The user may be presented with the information described above through a graphical user interface, such as through a drop down menu or list of patients and monitoring devices from which to select.

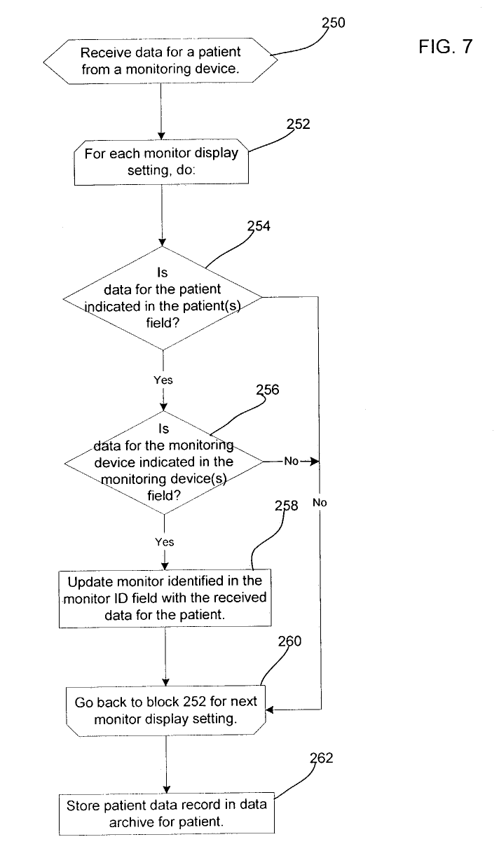

FIG. 7 illustrates operations performed by the monitor program 18 to display or render patient data received from one of the monitoring devices 4a, 4b, 6a, 6b according to the monitor display settings 100. Upon receiving (at block 250) patient data, a loop is performed at blocks 252 through 260 for each monitor display setting 80. If (at block 254) the data is for a patient indicated in the patient(s) field 104 of the monitor display setting 100 being considered and if (at block 256) the data is for a monitoring device indicated in the monitoring device(s) field 106 of the monitor display setting being considered, then the monitor program 18 updates (at block 258) the display monitor 20 identified in the display monitor ID field 102 of the monitor display setting 100 being considered with the received data for the patient. The received patient data record may further be stored (at block 262) in the data archive 20. Refreshing the display monitor with the received data for a given patient may not alter data displayed for other patients, and may only update any previously displayed data for the given patient.

Described implementations provide techniques for integrating the management of multiple bio-feedback monitoring devices that may be monitoring patients in a health care facility. With the described implementations, biometric feedback from monitoring devices from different medical device vendors may be gathered and managed together. Further, with the described implementations, the user may specify how data from a wide variety of devices is to be presented to the user on a display monitor and managed. Still further, a single user interface and monitoring program is used to manage biometric feedback from different patients and from medical devices from different vendors, even when the different vendor monitoring devices have proprietary components, to gather and transmit patient biometric data. In this way, the monitor program 18 may provide integrated management and control over biometric data from a wide array of different medical devices from different device vendors.

### Additional Implementation Details

The described techniques for managing a plurality of medical devices used to gather biometric data from patients in a health facility may be implemented as a method, apparatus or article of manufacture using standard programming and/or engineering techniques to produce software, firmware, hardware, or any combination thereof. The term “article of manufacture” as used herein refers to code or logic implemented in hardware logic (e.g., an integrated circuit chip, Programmable Gate Array (PGA), Application Specific Integrated Circuit (ASIC), etc.) or a computer readable medium, such as magnetic storage medium (e.g., hard disk drives, floppy disks,, tape, etc.), optical storage (CD-ROMs, optical disks, etc.), volatile and non-volatile memory devices (e.g., EEPROMs, ROMs, PROMs, RAMs, DRAMs, SRAMs, firmware, programmable logic, etc.). Code in the computer readable medium is accessed and executed by a processor. The code in which preferred embodiments are implemented may further be accessible through a transmission media or from a file server over a network. In such cases, the article of manufacture in which the code is implemented may comprise a transmission media, such as a network transmission line, wireless transmission media, signals propagating through space, radio waves, infrared signals, etc. Thus, the “article of manufacture” may comprise the medium in which the code is embodied. Additionally, the “article of manufacture” may comprise a combination of hardware and software components in which the code is embodied, processed, and executed. Of course, those skilled in the art will recognize that many modifications may be made to this configuration without departing from the scope of the present invention, and that the article of manufacture may comprise any information bearing medium known in the art.

In described implementations, a single management interface was described as gathering the data from different monitoring devices monitoring biometric data from multiple patients. In alternative implementations, multiple management interface systems may be deployed to gather biometric data from different monitoring devices, and transmit the gathered biometric data to the central monitoring system to manage.

In the described implementations, the client monitor executing in an embedded operating system of a monitoring device, the management interface, and sniffer device gather and further transmit biometric data to a downstream component. Each of these components may in turn further manipulate and process the biometric data before the biometric data reaches the monitoring program 18 in the form of a patient data record. Alternatively, the monitoring program in the client monitoring system may also further transform data into a format, such as a patient data record, that the monitoring program would then use.

In the described implementations, the monitoring program 18 was described as checking alert thresholds and performing alert related actions. In alternative implementations, any component can perform alert or data processing operations. For instance, the monitoring devices, sniffer devices or management interface may process alert events to determine whether an alert occurred based on the received biometric data exceeding a predefined alert threshold.

Described implementations provided certain fields used with a patient data record, alert notification, and monitor display setting. In further implementations, additional fields and data may be used with these data structures. Still further, an alternative arrangement of one or more data structures may be used to maintain the data described with respect to FIGS. 2, 3, and 4.

The illustrated operations in FIGS. 5, 6, and 7 show certain events occurring in a certain order. In alternative implementations, certain operations may be performed in a different order, modified or removed. Morever, steps may be added to the above described logic and still conform to the described implementations. Further, operations described herein may occur sequentially or certain operations may be processed in parallel. Yet further, operations may be performed by a single processing unit or by distributed processing units.

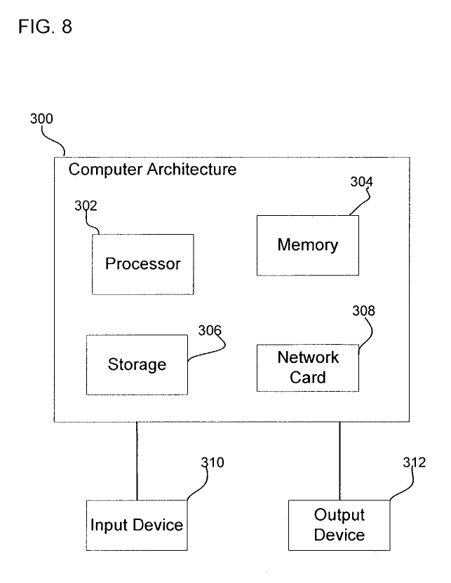

FIG. 8 illustrates one implementation of a computer architecture 300 of the systems, such as monitoring devices 4a, 4b, sniffer device 14a, 14b, management interface 12, and central monitoring system 16 shown in FIG. 1. The architecture 300 may include a processor 302 (e.g., a microprocessor), a memory 304 (e.g., a volatile memory device), and storage 306 (e.g., a non-volatile storage, such as magnetic disk drives, optical disk drives, a tape drive, etc.). The storage 306 may comprise an internal storage device or an attached or network accessible storage. Programs in the storage 306 are loaded into the memory 304 and executed by the processor 302 in a manner known in the art. The architecture further includes a network card 308 to enable communication with a network. An input device 310 is used to provide user input to the processor 302, and may include a keyboard, mouse, pen-stylus, microphone, touch sensitive display screen, or any other activation or input mechanism known in the art. An output device 312 is capable of rendering information transmitted from the processor 302, or other component, such as a display monitor, printer, storage, etc.

The foregoing description of various implementations of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto. The above specification, examples and data provide a complete description of the manufacture and use of the composition of the invention. Since many embodiments of the invention can be made without departing from the spirit and scope of the invention, the invention resides in the claims hereinafter appended.

What is claimed is:

1. A method for managing patient biometric data from a plurality of patients in a health care environment, comprising:
receiving different types of biometric data from a plurality of different types of medical monitoring devices;
determining from display settings one of a plurality of display monitors on which to display received biometric data from one of the plurality of medical monitoring devices; and
displaying the received biometric data on the determined monitor, wherein each monitor is capable of displaying biometric data from at least one patient and from at least one monitoring device.

2. The method of claim 1, wherein the different types of medical monitoring devices comprise medical devices that monitor different types of biometric data.

3. The method of claim 1, wherein the different types of medical monitoring devices comprise medical devices from different medical device vendors

4. The method of claim 3, wherein the biometric data is received from medical monitoring devices that use different data transmittal formats, and wherein displaying the biometric data comprises:
displaying biometric data on the determined monitor that is received from medical monitoring devices that use different data transmittal formats.

5. The method of claim 4, wherein a first set of the displayed biometric data is received from a medical monitoring device that includes an embedded operating system and client program that transmits data using an industry standard data transmittal format and wherein a second set of the displayed biometric data is received from a medical monitoring device that uses a proprietary data transmittal format.

6. The method of claim 1, wherein displaying the biometric data comprises:
displaying biometric data on the determined monitor that is received from different types of medical monitoring devices and different patients.

7. The method of claim 1, further comprising:
providing information for the display monitors indicating at least one patient and at least one monitoring device for one display monitor, wherein displaying the received biometric data comprises displaying on the display monitor the biometric data from the at least one monitoring device and for the at least one patient indicated for the display monitor.

8. The method of claim 1, further comprising:
storing biometric data received from different patients and different types of monitoring devices in a patient data archive.

9. The method of claim 1, further comprising:
determining whether the received biometric data satisfies a threshold related to an alert; and
performing an alert action related to the threshold if the received biometric data satisfies the threshold.

10. The method of claim 9, further comprising:
maintaining information on thresholds and related alert actions for different types of monitoring devices, wherein different alert actions are performed for different monitoring devices.

11. The method of claim 10, wherein the different types of monitoring devices transmit biometric data in different formats.

12. A system for managing patient biometric data from a plurality of patients in a health care environment, comprising:
means for receiving different types of biometric data from a plurality of different types of medical monitoring devices;
means for determining from display settings one of a plurality of display monitors on which to display received biometric data from one of the plurality of medical monitoring devices; and
means for displaying the received biometric data on the determined monitor, wherein each monitor is capable of displaying biometric data from at least one patient and from at least one monitoring device.

13. The system of claim 12, wherein the different types of medical monitoring devices comprise medical devices that monitor different types of biometric data.

14. The system of claim 12, wherein the different types of medical monitoring devices comprise medical devices from different medical device vendors

15. The system of claim 14, wherein the biometric data is received from medical monitoring devices that use different data transmittal formats, and wherein the means for displaying the biometric data performs:
displaying biometric data on the determined monitor that is received from medical monitoring devices that use different data transmittal formats.

16. The system of claim 15, wherein a first set of the displayed biometric data is received from a medical monitoring device that includes an embedded operating system and client program that transmits data using an industry standard data transmittal format and wherein a second set of the displayed biometric data is received from a medical monitoring device that uses a proprietary data transmittal format.

17. The system of claim 12, wherein the means for displaying the biometric data performs:
displaying biometric data on the determined monitor that is received from different types of medical monitoring devices and different patients.

18. The system of claim 12, further comprising:
means for providing information for the display monitors indicating at least one patient and at least one monitoring device for one display monitor, wherein the means for displaying the received biometric data displays on the display monitor the biometric data from the at least one monitoring device and for the at least one patient indicated for the display monitor.

19. The system of claim 12, further comprising:
means for storing biometric data received from different patients and different types of monitoring devices in a patient data archive.

20. The system of claim 12, further comprising:
means for determining whether the received biometric data satisfies a threshold related to an alert; and
means for performing an alert action related to the threshold if the received biometric data satisfies the threshold.

21. The system of claim 20, further comprising:
means for maintaining information on thresholds and related alert actions for different types of monitoring devices, wherein different alert actions are performed for different monitoring devices.

22. The system of claim 21, wherein the different types of monitoring devices transmit biometric data in different formats.

23. An article of manufacture for managing patient biometric data from a plurality of patients in a health care environment, wherein the article of manufacture performs operations, the operations comprising:
receiving different types of biometric data from a plurality of different types of medical monitoring devices;
determining from display settings one of a plurality of display monitors on which to display received biometric data from one of the plurality of medical monitoring devices; and
displaying the received biometric data on the determined monitor, wherein each monitor is capable of displaying biometric data from at least one patient and from at least one monitoring device.

24. The article of manufacture of claim 23, wherein the different types of medical monitoring devices comprise medical devices that monitor different types of biometric data.

25. The article of manufacture of claim 23, wherein the different types of medical monitoring devices comprise medical devices from different medical device vendors

26. The article of manufacture of claim 25, wherein the biometric data is received from medical monitoring devices that use different data transmittal formats, and wherein displaying the biometric data comprises:
displaying biometric data on the determined monitor that is received from medical monitoring devices that use different data transmittal formats.

27. The article of manufacture of claim 26, wherein a first set of the displayed biometric data is received from a medical monitoring device that includes an embedded operating system and client program that transmits data using an industry standard data transmittal format and wherein a second set of the displayed biometric data is received from a medical monitoring device that uses a proprietary data transmittal format.

28. The article of manufacture of claim 23, wherein displaying the biometric data comprises:
displaying biometric data on the determined monitor that is received from different types of medical monitoring devices and different patients.

29. The article of manufacture of claim 23, further comprising:
providing information for the display monitors indicating at least one patient and at least one monitoring device for one display monitor, wherein displaying the received biometric data comprises displaying on the display monitor the biometric data from the at least one monitoring device and for the at least one patient indicated for the display monitor.

30. The article of manufacture of claim 23, further comprising:
storing biometric data received from different patients and different types of monitoring devices in a patient data archive.

31. The article of manufacture of claim 23, further comprising:
determining whether the received biometric data satisfies a threshold related to an alert; and
performing an alert action related to the threshold if the received biometric data satisfies the threshold.

32. The article of manufacture of claim 31, further comprising:
maintaining information on thresholds and related alert actions for different types of monitoring devices, wherein different alert actions are performed for different monitoring devices.

33. The article of manufacture of claim 32, wherein the different types of monitoring devices transmit biometric data in different formats.
